(19) United States
(12) Patent Application Publication
(10) Pub. No.: US 2026/0137368 A1
(43) Pub. Date: May 21, 2026

(54) ULTRASOUND NAVIGATION SYSTEM WITH SIMPLIFIED PROBE POSITIONING INSTRUCTIONS

(71) Applicant: Ultrasight Ltd., Nes Ziona (IL)

(72) Inventor: Itay KEZURER, Rehovot (IL)

(21) Appl. No.: 19/386,344

(22) Filed: Nov. 12, 2025

Related U.S. Application Data
(60) Provisional application No. 63/721,589, filed on Nov. 18, 2024.

(51) Int. Cl. A61B 8/00

(52) U.S. Cl. CPC A61B 8/4254; A61B 8/4427; A61B 8/461; A61B 8/58

(57) ABSTRACT

An ultrasound navigation device includes a processor with a trained orientation neural network and a simplified motion result converter. The trained orientation neural network receives a sequence of input images of a body part from an ultrasound unit. It then generates a sequence of transformations that indicate the translation and rotation motions needed to move from the probe's current orientation to a selected canonical view. The simplified motion result converter converts the sequence of transformations into a simplified motion instruction for the probe. It displays this instruction to a user to guide a change in the probe's position or rotation. The converter contains a display logic that determines which motion from the transformation sequence has a value above an associated predetermined threshold. The display logic then presents this determined motion to the user as the simplified instruction.

### CROSS REFERENCE TO RELATED APPLICATIONS

This application claims priority from U.S. provisional patent application 63/721,589, filed Nov. 18, 2024, which is incorporated herein by reference.

### FIELD OF THE INVENTION

The present invention relates generally to ultrasound imaging systems and to guidance for positioning ultrasound probes in particular.

### BACKGROUND OF THE INVENTION

A medical ultrasound (also known as diagnostic sonography or ultrasonography) is a diagnostic imaging technique based on the application of an ultrasound. It is used to create an image of internal body structures such as tendons, muscles, joints, blood vessels and internal organs.

Acquiring accurate images in order to perform an effective examination and diagnosis requires placing the ultrasound transducer in an angular position in space with the pertinent organ or body part, as is illustrated in FIG. 1 to which reference is now made. FIG. 1 shows an ultrasound image of an organ 12 of interest taken with a transducer 14. It will be appreciated that the art of navigating transducer 14 to the exact angular position required to achieve the optimal or “canonical” image of organ 12 is crucial to the success of the ultrasound examination. The process typically requires a trained and skilled sonographer.

For example, in order to perform an echocardiogram, the sonographer has to take images of the heart from various canonical directions, such as four-chamber and two-chamber views. The correct positioning of the transducer is crucial to receiving the optimal view of the left ventricle and consequently to extract the functional information of the heart.

Mobile ultrasound machines or devices are known in the art, such as the Lumify commercially available from Philips. These mobile ultrasound machines are available in the form of a transducer that communicates with a program downloadable to any portable handheld device such as a smart phone or a tablet.

The availability of such devices means that ultrasounds may be performed off-site (away from hospitals, etc.) for example, as a triage tool for ambulances or even on the battlefield, at urgent care facilities, nursing homes, etc. without requiring bulky expensive equipment.

U.S. Pat. No. 11,593,638 describes a neural network-based ultrasound navigator which is useful for untrained doctors, first aid providers, or even patients to administer these ultrasounds correctly, using only an ultrasound probe. The navigator of U.S. Pat. No. 11,593,638 operates only with the digital images generated by the ultrasound unit and is described hereinbelow with reference to FIGS. 2-6.

Reference is now made to FIG. 2 which illustrates the ultrasound navigator 100 of U.S. Pat. No. 11,593,638, which may be downloaded from a mobile application store 10 onto any portable computing device. Thus, a user 5, which may or may not be skilled in the art of sonography, uses a transducer or probe 7 (associated with mobile ultrasound unit 8) on patient 9 to supply images of a pertinent body part to navigator 100 and navigator 100 supplies orientation instructions accordingly as to how to orientate probe 7, where “orientation” instructions comprise both position (location in two or three-dimensional space) and rotation information (rotation in 3D space), even though navigator 100 receives only images.

Reference is now made to FIGS. 3A and 3B which illustrate how navigator 100 aids non-sonographer 5 to orientate probe 7 in order to capture a good image of a particular body part. FIG. 3A shows probe 7, labeled 7A, in the wrong position, i.e. the resultant image, labeled 20A, is not canonical. FIG. 3A additionally includes a set of arrows 21 instructing user 5 to change the rotation of probe 7A. Arrows 21A indicate a ‘pitch up’ kind of rotation. FIG. 3B shows probe 7B in the newly pitched US orientation and the resultant image 20B, which is better, though still not providing a canonical image. Arrows 21B indicate a new “yaw” rotation may be useful.

Navigator 100 receives orientation neural network 15 which is trained with expert data taken by a skilled sonographer for a particular body part or organ of interest. The training data includes the canonical image of a particular body part as well as associated non-canonical images and for each, the orientation (i.e. position and rotation) of the sonographer's probe in space.

Reference is now made to FIG. 4 which illustrates the transformation between the orientation of a training probe 4c used by a trained sonographer for capturing the canonical image in relation to its orientation when capturing a non-canonical image for an organ. The orientation of training probe 4i when viewing the i^{th} non-canonical image is defined as a “frame of reference” Fᵢ in space where frame of reference Fᵢ has the six degrees of freedom (6DoF), corresponding to a three-axis system (Q) having three rotations around the axes and three translations along the axes, that an IMU may measure.

Frames of reference Fᵢ refer to frame of reference at an origin O, where the origin is at the organ and its frame of reference in space is defined as Fₒ. For each frame of reference Fᵢ, there is a transformation Rᵢ from the origin O, where the transformation R_{c} is a transformation to the desired orientation, labeled Fₑ, for viewing the canonical image, as follows:

$${R}_{c} = {F}_{c} {F}_{o}^{- 1} \qquad ( 1 )$$

$${R}_{i} = {F}_{i} {F}_{o}^{- 1}$$

where Fₒ⁻¹ is the inverse transform of Fₒ. Thus, a transformation Tᵢ from the canonical pose to the ith non-canonical pose is RᵢR_{c}⁻¹:

$${T}_{i} = {R}_{i} {R}_{c}^{- 1} = {F}_{i} {F}_{0}^{- 1} ( {F}_{o} {F}_{c}^{- 1} ) = {F}_{i} {F}_{c}^{- 1} \qquad ( 2 )$$

Reference is now made to FIG. 5 which illustrates the training process for orientation neural network 15 using a trainer 30. A skilled sonographer 2 using training probe 4 on a patient 3 provides both canonical and associated non-canonical images for a particular body part. Training probe 4 is associated with an IMU 6 (an inertial measurement unit which may include a magnetometer, a gyroscope, an accelerometer, etc.) which determines the orientation Fᵢ of the probe when an image is captured.

Training converter 22 receives the orientation data F; for each image and determines the transformation Tᵢ=RᵢR_{c}⁻¹ from the associated canonical position, as discussed herein above with respect to FIG. 4. Specifically, training converter 22 takes images X from training probe 4 and processes them as necessary. Database 20 stores non-canonical images X together with their orientation data Fᵢ and their transformation data Tᵢ. Database 20 also stores canonical images Xᵢ and their associated orientation data Fₑ.

Trainer 30 trains the network by updating the network to minimize an energy “loss” as determined by a loss function such as a distance between a calculated transformation S(Xᵢ) produced by orientation neural network 15 and the ground truth transformation Tᵢ for image Xᵢ from its associated canonical image. If there is more than one associated canonical image, orientation neural network 15 is trained with the ground truth transformation Tᵢ to each non-canonical image. A loss function “Loss” is calculated as:

$$Loss = loss ( S ( {X}_{i} ) , {T}_{i} ) \qquad ( 3 )$$

Once orientation neural network 15 is trained, it generates a transformation T for user probe 7 in response to each incoming image Xᵢ. This transformation is then converted to guide user 5 from the orientation for the non-canonical image to the orientation for the canonical image.

Reference is now made to FIG. 6 which illustrates the components of navigator 100. Navigator 100 comprises trained orientation neural network 15, a result converter 40 and a diagnoser 50.

User 5 randomly places user probe 7 in relation to the desired body part. Trained orientation neural network 15 provides the transformation T from the associated canonical image to the current non-canonical image of a particular body part. Result converter 40 converts the generated transformation to provide orientation instructions for probe 7 from the current position and rotation viewing a non-canonical image to a position and rotation to view the associated canonical image. Result converter 40 displays these orientation instructions to user 5 in various ways. The process is iterative until user 5 positions probe 7 correctly (within an error range).

Result converter 40 converts the orientation data S(X) produced by trained orientation neural network 15 into instructions to user 5 about how to maneuver probe 7 to achieve the selected canonical image. U.S. Pat. No. 11,593,638 shows one display with reference to FIGS. 3A and 3B, which displays rotation markings.

Diagnoser 50 receives the final canonical image produced by user 5 and detects any anomalies therein.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of how an ultrasound transducer is placed to capture an image of a body part;

FIG. 2 is a schematic illustration of a prior art ultrasound navigator;

FIGS. 3A and 3B are schematic illustrations of how the navigator of FIG. 2 may aid a non-sonographer orientate a probe in order to capture a suitable image of a body part;

FIG. 4 is a schematic illustration of the transformation between the orientation of a training probe for a non-canonical image of an organ and its associated canonical image;

FIG. 5 is a schematic illustration of the training process for an orientation neural network;

FIG. 6 is a schematic illustration of the elements of the navigator of FIG. 2;

FIG. 7 is a schematic illustration of an ultrasound navigator having a simplified motion result converter, constructed and operative in accordance with a preferred embodiment of the present invention;

FIG. 8 is schematic illustration of the transformation of FIG. 4 and the probe axis motions therein;

FIG. 9 is a mathematical illustration of the transformation of FIG. 8;

FIG. 10 is a flowchart illustration of a display logic forming part of the simplified motion result converter of FIG. 7; and

FIGS. 11A and 11B are exemplary motion displays produced by the simplified motion result converter of FIG. 7.

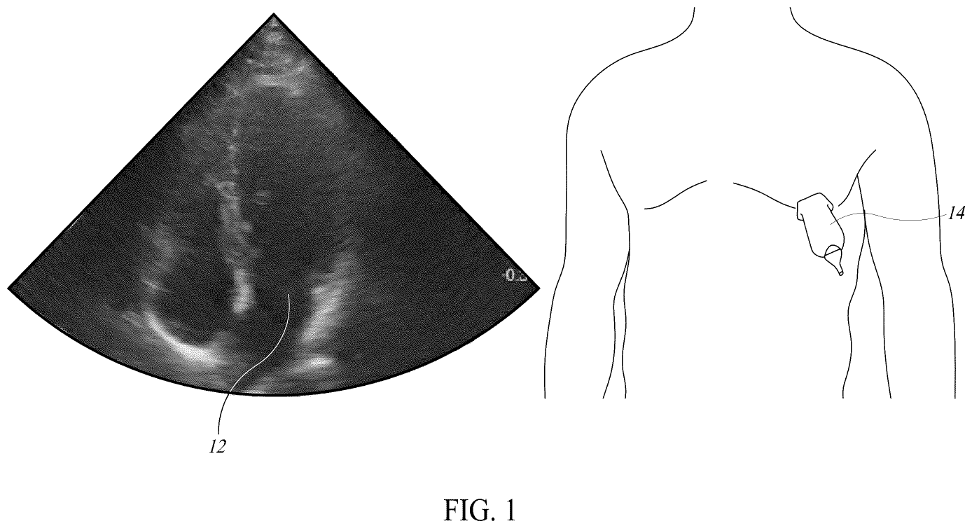

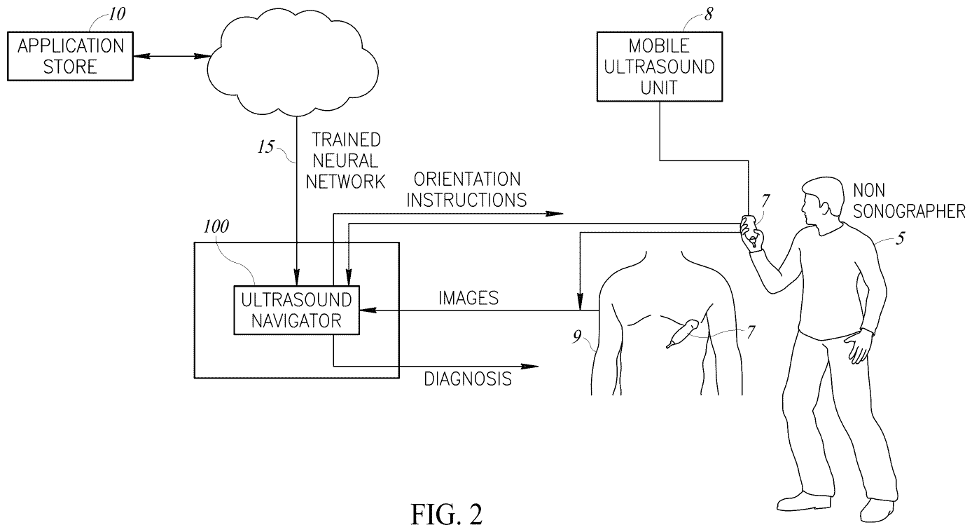

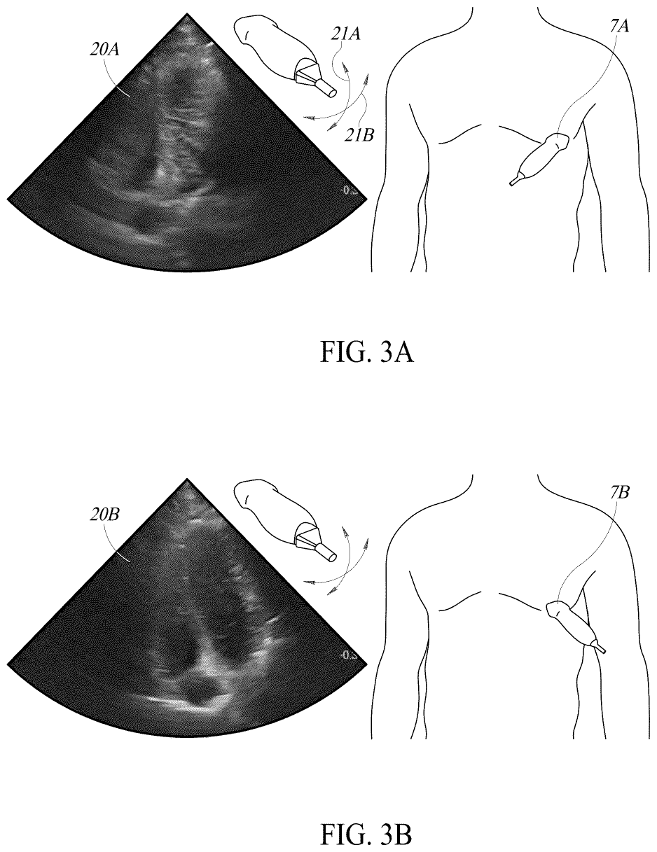

It will be appreciated that for simplicity and clarity of illustration, elements shown in the Figures have not necessarily been drawn to scale. For example, the dimensions of some of the elements may be exaggerated relative to other elements for clarity. Further, where considered appropriate, reference numerals may be repeated among the Figures to indicate corresponding or analogous elements.

### SUMMARY OF THE PRESENT INVENTION

There is therefore provided, in accordance with a preferred embodiment of the present invention, a device for an ultrasound unit having an ultrasound probe, the device including a processor. The processor includes a trained orientation neural network and a simplified motion result converter. The trained orientation neural network receives a sequence of input non-canonical and/or canonical images of a body part from the ultrasound unit and generates a sequence of transformations to transform between a position and rotation associated with a selected canonical view and a position and rotation associated with one of the input images, the sequence of transformations indicating a sequence of translation and rotation motions per input image to the selected canonical view. A simplified motion result converter converts the sequence of transformations into a simplified motion instruction for the probe and displays the simplified motion instruction to a user to change a position or rotation of the probe. The simplified motion result converter includes a display logic to determine which motion indicated by the sequence of transformations has at least one value above its associated predetermined threshold and displays the determined motion to the user as the simplified motion instruction.

Moreover, in accordance with a preferred embodiment of the present invention, the simplified motion instruction includes one of: a translation instruction along an x-axis of the probe, a translation instruction along a y-axis of the probe, a tilt rotation instruction around an x-axis of the probe, a rock rotation instruction around a y-axis of the probe, and a roll rotation instruction around a z-axis of the probe.

Further, in accordance with a preferred embodiment of the present invention, the display logic has a set of motions and an order in which to consider them.

Still further, in accordance with a preferred embodiment of the present invention, the order is translation, roll rotation, rock rotation and tilt rotation.

Additionally, in accordance with a preferred embodiment of the present invention, the display logic includes a determining logic to determine the simplified motion instruction by first checking for translation motions and then checking for rotation motions.

Moreover, in accordance with a preferred embodiment of the present invention, the simplified motion result converter includes a display unit to display the simplified motion instruction as a graphical representation on a display.

Further, in accordance with a preferred embodiment of the present invention, the display logic includes orientation achieved logic to identify when the sequence of transformations has no value above its associated predetermined threshold and, in response, to provide an orientation achieved indication.

There is therefore provided, in accordance with a preferred embodiment of the present invention, a method for an ultrasound unit having an ultrasound probe. The method includes receiving a sequence of input non-canonical and/or canonical images of a body part from the ultrasound unit, generating a sequence of transformations to transform between a position and rotation associated with a selected canonical view and a position and rotation associated with one of the input images, the sequence of transformations indicating a sequence of translation and rotation motions per input image to the selected canonical view. The method further includes converting the sequence of transformations into a simplified motion instruction for the probe, and displaying the simplified motion instruction to a user to change a position or rotation of the probe. The converting includes determining which motion indicated by the sequence of transformations has at least one value above its associated predetermined threshold, and where the determined motion is the simplified motion instruction.

Still further, in accordance with a preferred embodiment of the present invention, the simplified motion instruction includes one of: a translation instruction along an x-axis of the probe, a translation instruction along a y-axis of the probe, a tilt rotation instruction around an x-axis of the probe, a rock rotation instruction around a y-axis of the probe, and a roll rotation instruction around a z-axis of the probe.

Additionally, in accordance with a preferred embodiment of the present invention, the converting uses a set of motions and an order in which to consider them.

Moreover, in accordance with a preferred embodiment of the present invention, the order is translation, roll rotation, rock rotation and tilt rotation.

Further, in accordance with a preferred embodiment of the present invention, the converting includes determining the simplified motion instruction by first checking for translation motions and then checking for rotation motions.

Still further, in accordance with a preferred embodiment of the present invention, the simplified motion instruction is a graphical representation on a display.

Additionally, in accordance with a preferred embodiment of the present invention, the converting includes identifying when the sequence of transformations has no value above its associated predetermined threshold and, in response, providing an orientation achieved indication.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that providing complex instructions on how to move with respect to arbitrary axes, as taught by U.S. Pat. No. 11,593,638, can be overwhelming for inexperienced users and that providing one primary adjustment at a time, along one of the axes of the probe rather than along the arbitrary axis, may significantly simplify the user experience and may improve the efficiency of achieving canonical views for non-expert users.

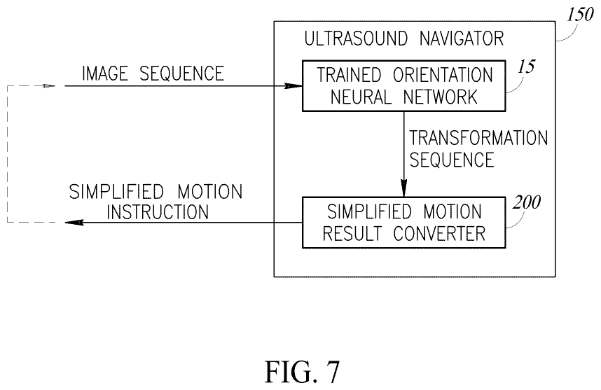

FIG. 7 illustrates a block diagram of an alternative ultrasound navigator system 150 which comprises trained orientation neural network 15 and a simplified motion result converter 200. Trained orientation neural network 15 may receive an input image sequence (which may be of non-canonical images if the user has not yet found a canonical view, or of canonical images, if the user is close to or at a selected canonical view) and may process these images to generate a transformation sequence, to the selected canonical view, as output. Simplified motion result converter 200 may receive the transformation sequence and may produce therefrom a simplified motion instruction (i.e. either a translation or a rotation around an axis of the probe) from the transformation sequence. The user then may move the probe in the simplified motion and the process repeats until the user brings the probe to the proper location for the selected canonical view.

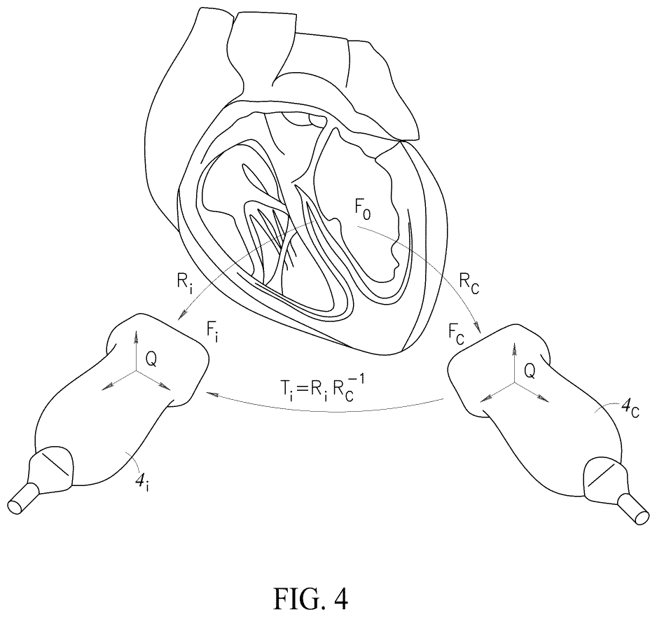

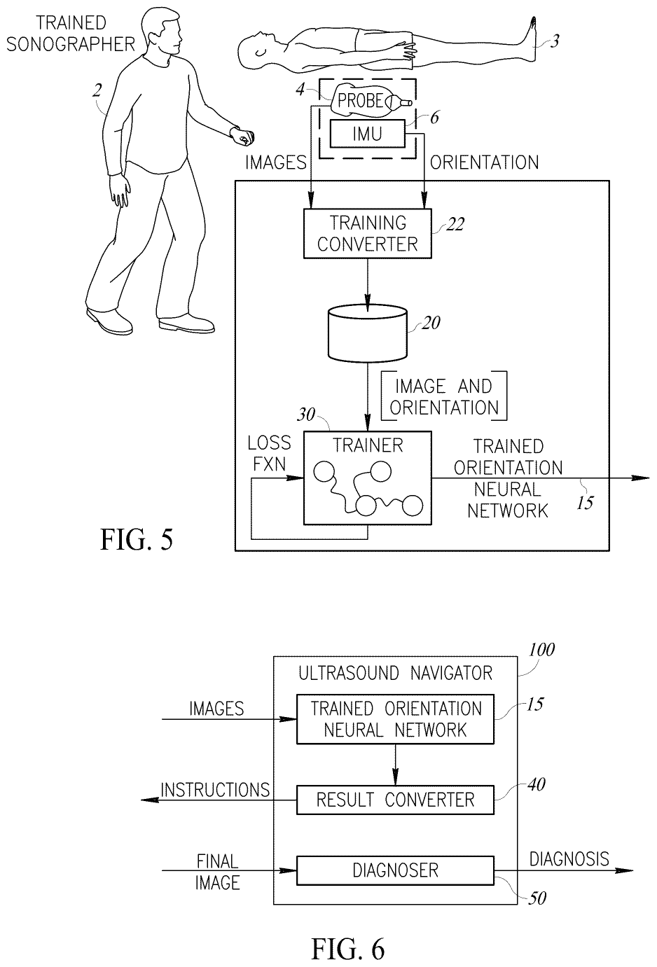

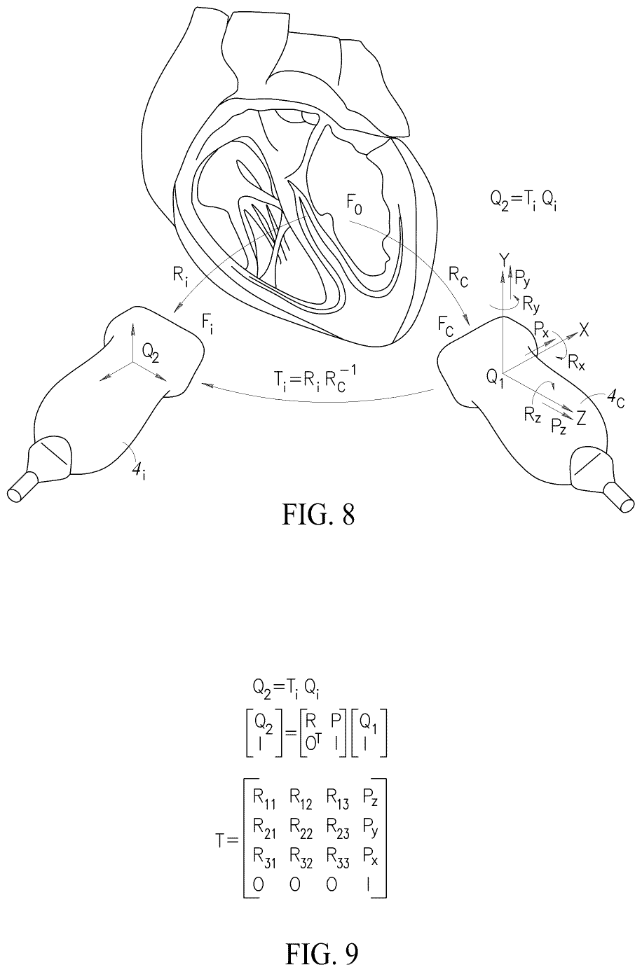

Reference is now made to FIG. 8, which is similar to FIG. 4 and illustrates the coordinate transformations produced by trained orientation neural network 15. Like FIG. 4, FIG. 8 depicts a heart with an ultrasound probe positioned at two different orientations relative to the heart.

FIG. 8 shows two coordinate frames, Q₁ and Q₂, associated with the two orientations, as well as origin frame F₀ and two additional frames, Fᵢ and Fₑ, representing different imaging orientations. Frame Fᵢ may correspond to a non-canonical image orientation, while frame Fₑ may represent a canonical image orientation. FIG. 8 also illustrates transformation Tᵢ between non-canonical and canonical orientations, where Q₂=TᵢQ₁.

As can be seen in FIGS. 8, Q₁ and Q₂ are coordinate systems aligned along the main axes (i.e. “probe axes”) of probes 4c and 4i, respectively. In FIG. 8, the axes of Q₁ are labeled x, y, and z, where the z axis is along the longitudinal axis of probe 4c, a translation along one of the axes is defined as Pₓ, P_{y}, or P_{z}, and the rotations around the axes are labeled Rₓ, R_{y}, and R_{z}, where R_{z} is a roll rotation, R_{y} is a rock rotation and Rₓ is a tilt rotation. As will be described hereinbelow, result converter 200 may generate simplified motion instructions along and around these axes. Note that the positive z axis is vertically away from the patient's body.

Reference is now made to FIG. 9, which illustrates the structure of transformation Ti when transforming from coordinate frame Q₁ to coordinate frame Q₂. As provided in equation 4 hereinbelow, transformation matrix Tᵢ may be a 4×4 matrix that combines a 3×3 rotation matrix R and a 3×1 translation vector P such that the transformation is a sum of a rotation and a translation. Thus, transformation matrix Tᵢ also includes a fourth row enabling the sum.

$$[ \begin{matrix}{Q}_{2} \\ 1\end{matrix} ] = [ \begin{matrix}R & P \\ {0}^{T} & 1\end{matrix} ] [ \begin{matrix}{Q}_{1} \\ 1\end{matrix} ] \qquad ( 4 )$$

Specifically, rotation matrix R may consist of elements R₁₁, R₁₂, R₁₃, R₂₁, R₂₂, R₂₃, R₃₁, R₃₂, and R₃₃, where the diagonal elements are R₁₁, R₂₂ and R₃₃. Applicant has realized that, when the diagonal elements R₁₁, R₂₂ and R₃₃ are larger than predefined thresholds, then the diagonal elements indicate rotations around the main axes of Q₂, where R11 indicates a roll rotation around the z axis, R22 indicates a rock rotation around the y axis and R33 indicates a tilt rotation around the x axis. Translation vector P may be composed of elements P_{z}, P_{y}, and Pₓ.

Accordingly, transformation matrix Tᵢ, when written in full, may be structured as follows:

$${T}_{i} = [ \begin{matrix}{R}_{11} & {R}_{12} & {R}_{13} & {P}_{z} \\ {R}_{21} & {R}_{22} & {R}_{23} & {P}_{y} \\ {R}_{31} & {R}_{32} & {R}_{33} & {P}_{x} \\ 0 & 0 & 0 & 1\end{matrix} ] \qquad ( 5 )$$

where the upper-left 3×3 submatrix may contain rotation matrix elements R₁₁ through R₃₃ and the rightmost column may contain translation vector elements P_{z}, P_{y}, and Pₓ. The bottom row may contain elements 0, 0, 0, and 1, which may enable the rotation action to be added to the translation action.

As described in more detail hereinbelow, result converter 200 may determine whether any of the rotations R or the translations P, in the x or y axes only, are large enough (i.e. each with respect to a predefined threshold for that motion) to display that motion to the user. Moreover, as described hereinbelow, result converter 200 may select the strongest motion (i.e. the motion largest above its threshold) in transformation matrix Tᵢ and may provide the associated instruction for it.

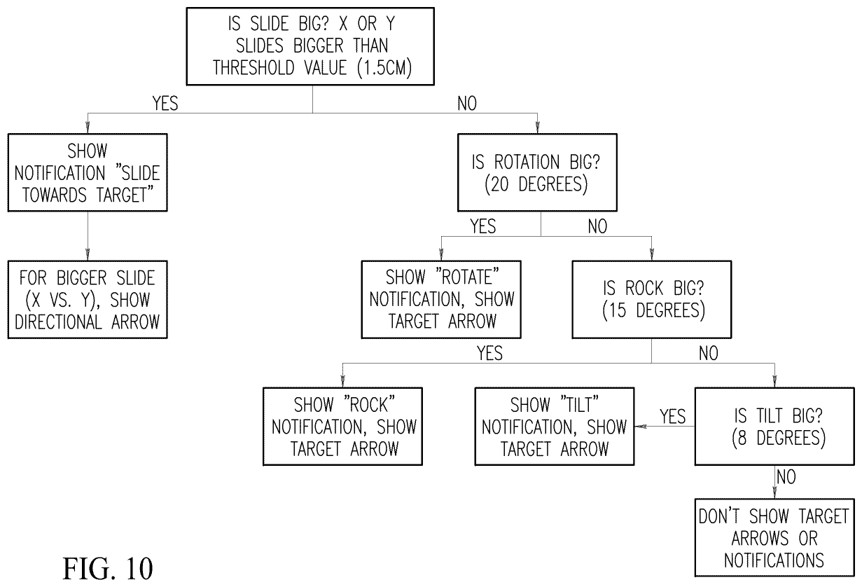

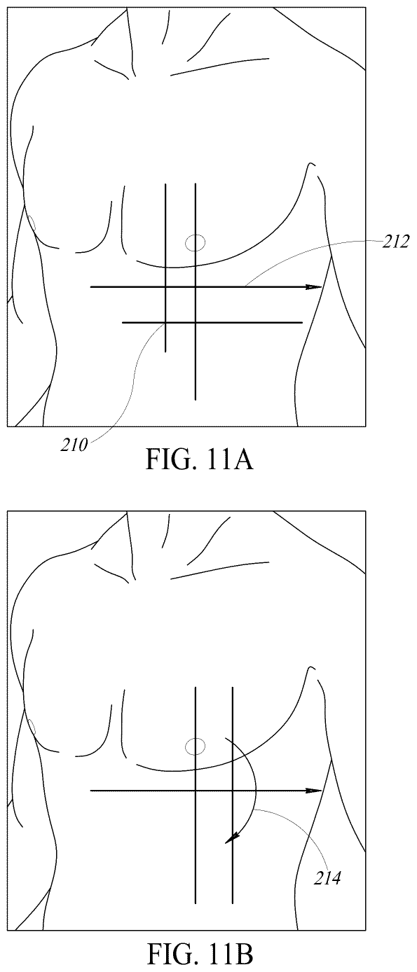

Reference is now made to FIG. 10, which illustrates a flowchart of a display logic for simplified motion result converter 200, and to FIGS. 11A and 11B, which are exemplary displays to the user for a translation motion and a rotation motion, respectively. For each sequence of transformations, result converter 200 may work through the display logic, starting with the translation part of the transformations Tᵢ in the transformation sequence and moving through the rotation part of the transformations Tᵢ. It will be appreciated that, by beginning with the translation motion, result converter 200 may initially have the user bring probe 7 close to the desired location and only after that, may instruct the user to maneuver probe 7 to the right angle for viewing the selected canonical view.

With respect to translation, result converter 200 may determine whether Pₓ or P_{y} has a sufficiently strong motion value (i.e. above its threshold which, in one exemplary embodiment, may be 1.5 cm) for at least one transformation in the transformation sequence. Note that, in most embodiments, P_{z} is not checked since motion along it would mean moving towards or away from the patient's body. However, embodiments providing instructions for such a motion are possible and are considered part of the present invention.

If Pₓ or P_{y} has a sufficiently strong motion value, result converter 200 may provide an instruction to the user to move probe 7 towards a crosshair 210 (FIG. 11A) indicating the correct position. FIG. 11A shows 2 crosshairs, crosshair 210 indicating the correct position and crosshair 212 indicating the current position of probe 7. During the following motion, result converter 200 may provide an indication, such as by changing a display color, when the user has brought probe 7 to or very close to the correct location (i.e. when the two crosshairs 210 and 212 overlap).

If none of the translation portions of the transformation sequence are large enough (which may typically occur once crosshairs 210 and 212 overlap), result converter 200 may check the various rotation angles in the transformation sequence against their predefined thresholds, in following order: rotation (or roll) value (i.e. R₁₁), then rock value (i.e. R₂₂) and then tilt value (i.e. R₃₃), of the transformation sequence. FIG. 10 lists exemplary thresholds for each rotation angle.

For whichever is the largest rotation angle, result converter 200 may provide an instruction (such as curved arrow 214 of FIG. 11B) indicating how the user should rotate probe 7. During the following motion, result converter 200 may provide an indication when the user has brought probe 7 to or very close to the correct rotation angle.

When none of the rotation angles in the transformation sequence are large enough (which may typically occur once probe 7 is viewing the selected canonical angle), result converter 200 may provide some indication that the desired orientation has been achieved.

Result converter 200 may display instructions, or notifications, in any suitable manner, such as with arrows and/or with text. Instructions and/or notifications may be displayed in any suitable location on a display of ultrasound navigator 100.

Unless specifically stated otherwise, as apparent from the preceding discussions, it is appreciated that, throughout the specification, discussions utilizing terms such as “analyzing,” “generating,” “processing,” “computing,” “calculating,” “determining,” or the like, refer to the action and/or processes of a general purpose computer of any type, such as a client/server system, mobile computing devices, smart appliances, cloud computing units or similar electronic computing devices that manipulate and/or transform data within the computing system's registers and/or memories into other data within the computing system's memories, registers or other such information storage, transmission or display devices.

Trained orientation neural network 15 and simplified motion result converter 200 may be implemented, together or separately, on any suitable apparatus. These apparatuses may be specially constructed for the desired purposes, or they may comprise a computing device or system typically having at least one processor and at least one memory, selectively activated or reconfigured by a computer program stored in the computer. The resultant apparatus when instructed by software may turn the general-purpose computer into inventive elements as discussed herein. The instructions may define the inventive device in operation with the computer platform for which it is desired. Such a computer program may be stored in a computer readable storage medium, such as, but not limited to, any type of disk, including optical disks, magnetic-optical disks, read-only memories (ROMs), volatile and non-volatile memories, random access memories (RAMs), electrically programmable read-only memories (EPROMs), electrically erasable and programmable read only memories (EEPROMs), magnetic or optical cards, Flash memory, disk-on-key or any other type of media suitable for storing electronic instructions and capable of being coupled to a computer system bus. The computer readable storage medium may also be implemented in cloud storage.

Some general-purpose computers may comprise at least one communication element to enable communication with a data network and/or a mobile communications network.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the invention as described herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A device for an ultrasound unit having an ultrasound probe, the device comprising:
a processor comprising:
a trained orientation neural network to receive a sequence of input non-canonical and/or canonical images of a body part from said ultrasound unit and to generate a sequence of transformations to transform between a position and rotation associated with a selected canonical view and a position and rotation associated with one of said input images, said sequence of transformations indicating a sequence of translation and rotation motions per input image to said selected canonical view; and
a simplified motion result converter to convert said sequence of transformations into a simplified motion instruction for said probe and to display said simplified motion instruction to a user to change a position or rotation of said probe, said simplified motion result converter comprising:
a display logic to determine which motion indicated by said sequence of transformations has at least one value above its associated predetermined threshold and to display said determined motion to said user as said simplified motion instruction.

2. The device of claim 1, wherein said simplified motion instruction comprises one of: a translation instruction along an x-axis of said probe, a translation instruction along a y-axis of said probe, a tilt rotation instruction around an x-axis of said probe, a rock rotation instruction around a y-axis of said probe, and a roll rotation instruction around a z-axis of said probe.

3. The device of claim 1 wherein said display logic has a set of motions and an order in which to consider them.

4. The device of claim 3 wherein said order is translation, roll rotation, rock rotation and tilt rotation.

5. The device of claim 2, wherein said display logic comprises a determining logic to determine said simplified motion instruction by first checking for translation motions and then checking for rotation motions.

6. The device of claim 1, wherein said simplified motion result converter comprises a display unit to display said simplified motion instruction as a graphical representation on a display.

7. The device of claim 1, wherein said display logic comprises orientation achieved logic to identify when said sequence of transformations has no value above its associated predetermined threshold and, in response, to provide an orientation achieved indication.

8. A method for an ultrasound unit having an ultrasound probe, the method comprising:
receiving a sequence of input non-canonical and/or canonical images of a body part from said ultrasound unit;
generating a sequence of transformations to transform between a position and rotation associated with a selected canonical view and a position and rotation associated with one of said input images, said sequence of transformations indicating a sequence of translation and rotation motions per input image to said selected canonical view; and
converting said sequence of transformations into a simplified motion instruction for said probe; and
displaying said simplified motion instruction to a user to change a position or rotation of said probe,
wherein said converting comprises determining which motion indicated by said sequence of transformations has at least one value above its associated predetermined threshold, and
wherein said determined motion is said simplified motion instruction.

9. The method of claim 8, wherein said simplified motion instruction comprises one of: a translation instruction along an x-axis of said probe, a translation instruction along a y-axis of said probe, a tilt rotation instruction around an x-axis of said probe, a rock rotation instruction around a y-axis of said probe, and a roll rotation instruction around a z-axis of said probe.

10. The method of claim 8 wherein said converting uses a set of motions and an order in which to consider them.

11. The method of claim 10 wherein said order is translation, roll rotation, rock rotation and tilt rotation.

12. The method of claim 9, wherein said converting comprises determining said simplified motion instruction by first checking for translation motions and then checking for rotation motions.

13. The method of claim 8, wherein said simplified motion instruction is a graphical representation on a display.

14. The method of claim 8, wherein said converting comprises identifying when said sequence of transformations has no value above its associated predetermined threshold and, in response, providing an orientation achieved indication.
